(19) United States
(12) Patent Application Publication
(10) Pub. No.: US 2011/0306924 A1
(43) Pub. Date: Dec. 15, 2011

(54) ATMOSPHERIC LOW-TEMPERATURE MICRO PLASMA JET DEVICE FOR BIO-MEDICAL APPLICATION

(76) Inventors: Sang Sik Yang, Seoul (KR); Kang II Kim, Jeollabuk-do (KR); Geun Young Kim, Gyeonggi-do (KR)

(21) Appl. No.: 13/203,693

(22) PCT Filed: Sep. 17, 2009
(86) PCT No.: PCT/KR09/05282

§ 371 Date: Aug. 26, 2011

(30) Foreign Application Priority Data
Feb. 27, 2009 (KR) 10-2009-0016663
Apr. 2, 2009 (KR) 10-2009-0028661

(51) Int. Cl. A61M 37/00; H01J 1/46; H05H 1/00; C25D 5/02

(52) U.S. Cl. 604/23; 205/122; 313/348; 313/231.31

(57) ABSTRACT

There are provided an atmospheric low-temperature micro plasma jet device for bio-medical application comprising an electrode used as an anode, a gas injection pipe used as a cathode, a porous insulating material, a protection pipe, and an insulating case and manufacturing method therefor using micromachining such as microelectromechanical systems (MEMS) in such a way that a diameter of micro electrodes where plasma is jetted is several tens micrometers or less.

### CROSS-REFERENCE TO RELATED APPLICATIONS

This application is a national phase application of PCT Application No. PCT/KR2009/005282 filed on Sep. 17, 2009, which claims priority to Korean Patent Application No. 10-2009-0028661 filed Apr. 2, 2009, and to Korean Patent Application No. 10-2009-0016663 filed on Feb. 27, 2009. The entire disclosures of the applications identified in this paragraph are incorporated herein by references.

### TECHNICAL FIELD

The present invention relates to an atmospheric low-temperature micro plasma jet device for bio-medical application, and more particularly, to a plasma jet device manufactured by using a micromachining process such as microelectromechanical systems (MEMS) in such a way that the diameter of micro electrodes jetting plasma is several tens of micrometers or less, thereby generating and jetting low-temperature plasma with a high current density using a low voltage under atmospheric pressure, which is capable of being applied to be in the field of bio-medical.

### BACKGROUND ART

Plasma has been applied to various fields such as semiconductor industry, display industry, and surface modification of materials. As plasma technology has been more and more developed, research for applying plasma to the medical field is proceeding. Plasma may be divided into high-temperature plasma and low-temperature plasma. When using high-temperature plasma for medical purpose, there occurs thermal damage to a cell. Accordingly, it is required for medical purposes to use a glow discharge that is low-temperature plasma. Since a glow discharge is very unstable under atmospheric pressure, the glow discharge is easily transited into an arc discharge that is high-temperature plasma.

To prevent glow to arc transition (GAT), heating on an electrode has to be prevented while a discharge occurs. In a way where a discharge occurs while gas continuously flows in, an electrode is naturally cooled, thereby generating a glow discharge stable under atmospheric pressure. There has been reported research on generating a discharge using a pipe or needle, mechanically processed, to generate a stable glow discharge under atmospheric pressure. However, there is a limitation on reducing a size via mechanical processing and it is difficult to process a broad area.

### DISCLOSURE OF INVENTION

### Technical Problem

The present invention provides a plasma jet device and an electrode used in the plasma jet device, manufactured by micromachining such as microelectromechanical systems (MEMS) in such a way that a diameter of micro electrodes where plasma is jetted is several tens of micrometers or less, thereby generating and jetting low-temperature plasma with a high current density using a low voltage under atmospheric pressure. This invention is applicable to the bio-medical field.

The present invention also provides a method of manufacturing the electrode used in a plasma jet device.

### Solution to Problem

According to an aspect of the present invention, there is provided a plasma jet device including an electrode used as an anode, a gas injection pipe used as a cathode, a porous insulating material, a protection pipe, and an insulating case. The electrode jets plasma. The gas injection pipe injects gas from the outside. The porous insulating material between the electrode and the gas injection pipe insulates the electrode from the gas injection pipe and includes a plurality of passing holes allowing the gas injected by the gas injection pipe to be passed to the electrode. The protection pipe surrounds the gas injection pipe to insulate and protect the gas injection pipe from the outside. The insulating case surrounds the porous insulating material to which the electrode and the gas injection pipe are connected and prevents the diffusion of a discharge occurring to generate the plasma between the electrode and the gas injection pipe. On the other hand, the gas injection pipe may be formed of stainless steel. On the other hand, the porous insulating material may be formed of ceramic, and more particularly, of alumina. On the other hand, the protection pipe may be formed of quartz. On the other hand, the plasma may be used for killing a cell where the plasma is jetted. In this case, the killed cell may be a cancer cell.

According to another aspect of the present invention, there is provided a method of manufacturing an electrode of a plasma jet device, the method including: forming a seed layer on a board; forming a mold layer on the seed layer; patterning the mold layer to form a plurality of electrode-forming holes thereon; forming an electrode layer on the board where the patterned mold layer is formed; and planarizing the patterned mold layer and the electrode layer; and removing the board, the seed layer, and the patterned mold layer. In forming a seed layer on a board, the seed layer is formed by depositing titanium/gold to a thickness of 500 Å and 2500 Å. In this case, the titanium/gold may be formed in a way of sputtering. In forming a mold layer on the seed layer, the mold layer is formed by coating with a negative sensitizer. In this case, a thickness of the mold layer may be 100 μm or less. In patterning the mold layer, the mold layer is patterned in such a way that each of the plurality of electrode-forming holes is disposed to be separated to one another with the same interval. In this case, a width of the electrode-forming holes may be 100 μm or less. Also, the number of the generated electrode-forming holes may be 10×10 or more. In forming an electrode layer, the electrode layer is formed by plating with a nickel layer. In this case, a thickness of the nickel layer formed on the electrode-forming holes may be 70 μm or less. In planarizing the patterned mold layer and the electrode layer, the patterned mold layer and the electrode layer are planarized in a way of chemical mechanical polishing (CMP). In this case, a thickness of the planarized electrode layer may be 60 μm or less.

### ADVANTAGEOUS EFFECTS OF INVENTION

As described above, a plasma jet device according to an embodiment of the present invention is manufactured by using a micromachining process such as microelectromechanical systems (MEMS) in such a way that a diameter of micro electrodes where plasma is jetted is several micrometers or less, thereby generating and jetting low-temperature plasma with a high current density using a low voltage under atmospheric pressure.

Also, thanks to the capability of jetting low-temperature micro plasma, the plasma jet device may be applied to the field of bio-medical using apoptosis.

### BRIEF DESCRIPTION OF DRAWINGS

FIG. 1 is a diagram illustrating a plasma jet device according to an embodiment of the present invention;

FIG. 2 is a cross-sectional view illustrating the plasma jet device of FIG. 1;

FIG. 3 is a flowchart illustrating a process of manufacturing an electrode of the plasma jet device of FIG. 1 using micromachining technology;

FIG. 4 is a diagram illustrating an example of an electrode manufactured according to the process of FIG. 3;

FIG. 5 is a graph illustrating a discharge firing voltage according to a gas flow rate in the plasma jet device of FIGS. 1; and

FIG. 6 is a graph illustrating discharge voltage/current characteristics according to a gas flow rate in the plasma jet device of FIG. 1.

### BEST MODE FOR CARRYING OUT THE INVENTION

To fully understand advantages of operations of the present invention and the objects obtained by embodiments of the present invention, it is required to refer to attached drawings illustrating preferable embodiments of the present invention and contents shown in the drawings. Hereinafter, the preferable embodiments of the present invention will be described in detail with reference to the attached drawings. The same reference numerals shown in each drawing indicate the same elements.

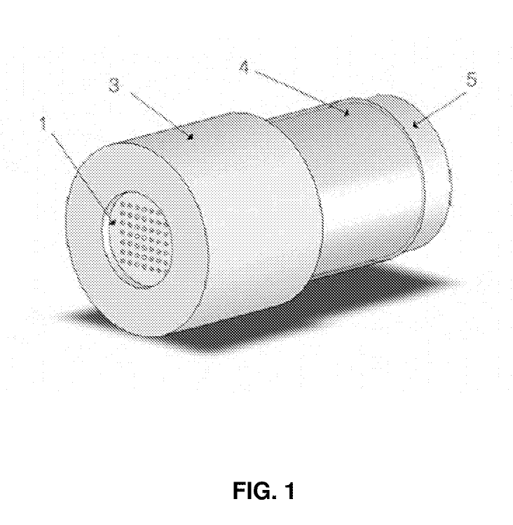

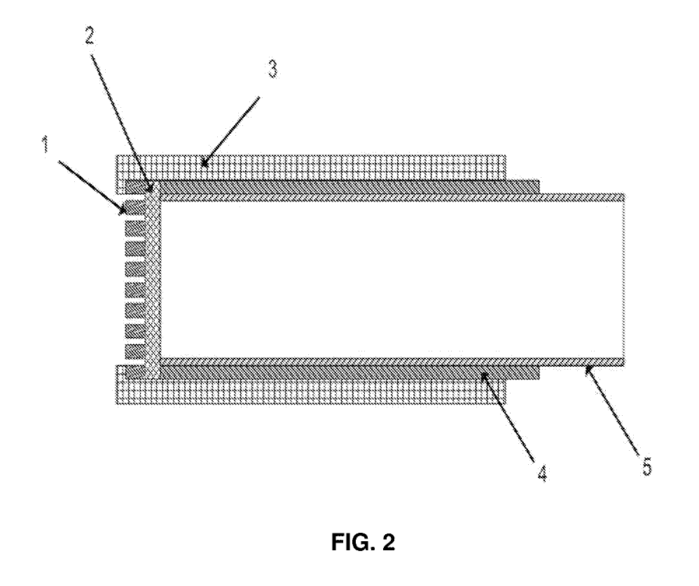

FIG. 1 is a diagram illustrating a plasma jet device according to an embodiment of the present invention. FIG. 2 is a cross-sectional view illustrating the plasma jet device of FIG. 1. The plasma jet device includes an electrode 1 used as an anode, a gas injection pipe 5 used as a cathode, a porous insulating material 2, a protection pipe 4, and an insulating case 3. There are a plurality of holes formed in the electrode 1, through which plasma generated by a discharge between the electrode 1 and the gas injection pipe 5 is jetted. In the present embodiment, the electrode 1 is formed of a metal, and more preferably, of nickel. The gas injection pipe 5 injects gas from the outside. In the present embodiment, the gas injection pipe 5 may be formed of stainless steel. The porous insulating material 2 is disposed between the electrode 1 and the gas injection pipe 5 and insulates the electrode 1 from the gas injection pipe 5. Also, the porous insulating material 2 allows the gas injected by the gas injection pipe 5 to be transported to the electrode 1 and may have a plurality of tubes and holes for this. In the present embodiment, the porous insulating material 2 may be formed of a ceramic material, and more preferably, formed of an alumina material.

The protection pipe 4 surrounds the gas injection pipe 5, thereby insulating and protecting the gas injection pipe 5 from the outside. In the present embodiment, the protection pipe 4 may be formed of a ceramic material, for example, quartz. The insulating case 3 surrounds the porous insulating material 2 to which the electrode 1 and the gas injection pipe 5 are connected. A discharge occurs between the electrode 1 and the gas injection pipe 5 to generate plasma. The insulating case 3 prevents such discharge from being diffused outside.

A theory of generating and jetting plasma in the plasma jet device is as follows. Gas flowing through the gas injection pipe 5 is ionized by an electric field formed between the holes of the electrode 1 and the gas injection pipe 5 while passing through the porous insulating material 2, thereby generating plasma. The plasma formed as described above is pushed out by gas injected by the gas injection pipe 5 and jetted via the holes of the electrode 1.

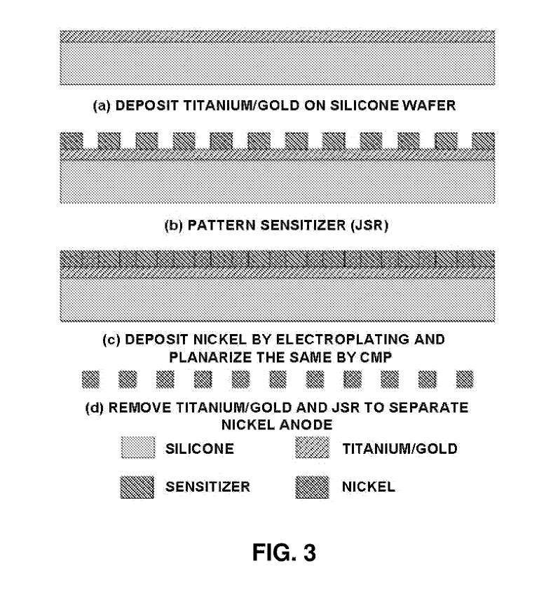

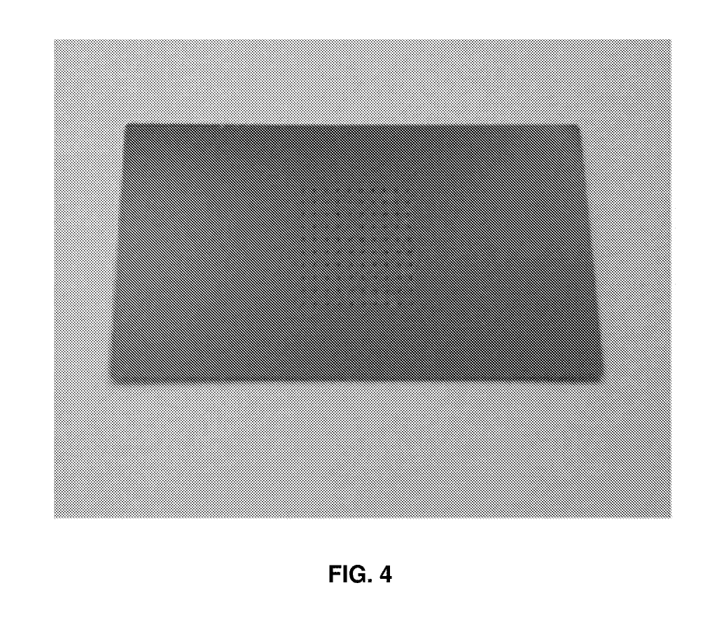

Hereinafter, referring to FIGS. 3 and 4, a process of manufacturing the electrode 1 of the plasma jet device of FIG. 1 will be described. FIG. 3 is a flowchart illustrating a process of manufacturing the electrode 1 of the plasma jet device of FIG. 1 using micromachining technology, and FIG. 4 is a diagram illustrating an example of an electrode manufactured by the process of FIG. 3.

Referring to FIG. 3(a), a seed layer is formed on a board. In the present embodiment, the board may be a silicone wafer. The seed layer may be formed by sputtering titanium/gold to be deposited. After forming the seed layer, a mold layer is formed on the seed layer. The mold layer will be used as a mold for electroplating to form an electrode layer that will be described later. In the present embodiment, the mold layer may be formed by coating the board with JSR that is a negative sensitizer. Also, the mold layer may be formed with a thickness of 100 μm or less. After forming the mold layer, the mold layer is patterned to form a plurality of electrode-forming holes thereon. The electrode layer that will be described later is formed on the electrode-forming holes. In this case, a width of the electrode-forming holes, such as a diameter of a cross-section thereof, may be 100 μm or less. Also, the number of the electrode-forming holes may be 10×10 or more in the electrode 1.

As shown in (b) of FIG. 3, in the present embodiment, the mold layer is formed corresponding to the holes of the electrode 1 shown in FIG. 1. In this case, the mold layer may be patterned in such a way that the plurality of electrode-forming holes are separated from one another with the same interval. Also, the mold layer may be patterned in such a way that holes corresponding to the patterned mold layer are separated from one another with several tens of micrometers.

After patterning the mold layer, as shown in (c) of FIG. 3, an electrode layer is formed on the board where the patterned mold layer is formed. In the present embodiment, the electrode layer may be formed by electroplating with a metal. The electroplated metal may be nickel. Also, a thickness of a nickel layer formed on the electrode-forming holes may be 70 μm or less. After forming the electrode layer, the patterned mold layer and the electrode layer are planarized and, as shown in (d) of FIG. 3, the board, the seed layer, and the patterned mold layer are removed, thereby forming the electrode 1 (referring to FIG. 4). In the present embodiment, planarization may be performed by chemical mechanical polishing. A thickness of the planarized electrode layer may be 60 μm or less. On the other hand, the plasma jet device according to an embodiment of the present invention may be used in the bio-medical field, and more particularly, for cure diseases by inducing apoptosis.

There are two mechanisms of the death of cells, such as necrosis and apoptosis. Necrosis is a way that a cell dies due to an external shock without its intention. Since the cell bursts and contaminates peripheral cells in this case, necrosis is not effective as medical treatment. On the other hand, apoptosis is a way that a cell kills itself. In this case, the cell does not contaminate peripheral cells in such a way that the problem of necrosis does not occur. Via researches in the bio-medical field, it is known that the cell kills itself when it is treated with plasma; that is, plasma is irradiated to the cell.

The plasma jet device according to an embodiment of the present invention may be used for medical treatment based on apoptosis mechanism. That is, the plasma jet device may be used for the purpose of treating diseases by jetting generated plasma to cells, such as cancer cells, to die.

### Embodiment 1

### Forming a Nickel Anode as an Electrode

A process of manufacturing a nickel anode was as follows. Titanium and gold, which would be seed layers, were deposited on a silicone board with 500 Å and 2500 Å. SU8-2100 that was a thick negative sensitizer was patterned to a thickness of 100 μm was used as a plating mold. Nickel plating employed nickel sulfamate baths. The nickel sulfamate baths was composed of 450 g/L of nickel sulfate [Ni(NH₂SO₃)₂₄H₂O], 30 g/L of boric acid added to reduce the stress of nickel, and 5 g/L of a humectant such as dodecyl sulfate sodium salt wetter to increase the quality of plated nickel. A nickel layer with a thickness of 70 μm was formed by plating for 80 hours at a current density of 1.3 mA/cm². To planarize the manufactured nickel layer, the thickness of the nickel layer was reduced to 60 μm by a chemical mechanical polishing (CMP) process. To separate the manufactured nickel layer, the silicone board was removed and SU8-2100 used as the mold was removed, thereby forming an anode.

### Embodiment 2

### Manufacturing a Plasma Jet Device

A plasma jet device included an anode, through which plasma is jetted, a dielectric layer insulating the anode from a cathode, and the cathode, into which gas flows. The anode was manufactured using nickel and a thickness thereof was 60 μm. The diameter of the hole, through which plasma is jetted, was 100 μm and a number thereof was 10×10. The dielectric layer was manufactured using porous alumina capable of insulating the anode from the cathode simultaneously while allowing the gas to pass. The thickness of the dielectric layer between the anode and the cathode was 1 mm. For the cathode, there was used a stainless steel tube with an external diameter of 1.6 mm and an internal diameter of 1.2 mm. For the sake of safety during a discharge experiment, the cathode was put into a quartz tube to insulate it from the surroundings thereof.

### Experimental Example 1

### Discharge Firing Voltage

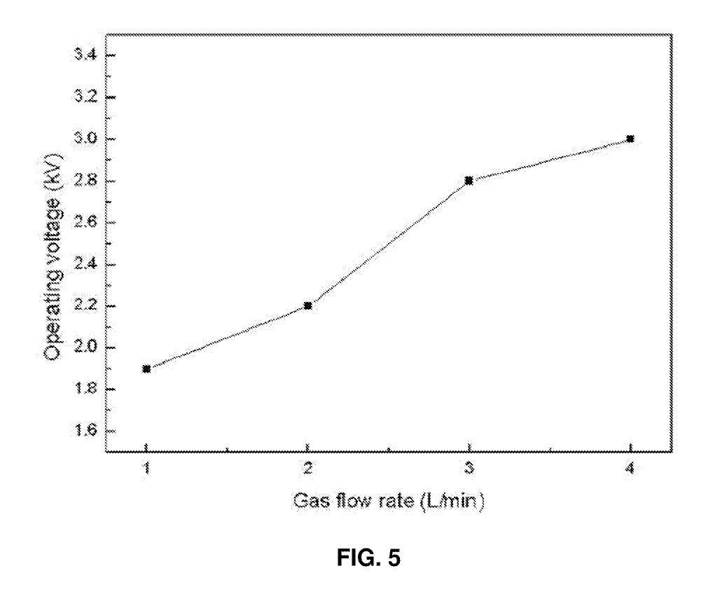

A discharge experiment was performed by using a nitrogen gas with a direct current (DC) under atmospheric pressure. A safety resistor of 2 MΩ was used and a voltage of 0 V to 9 kV was applied. To examine the effect of the flow rate of gas upon a discharge firing voltage, discharge characteristics depending on the flow rate were observed. A temperature of plasma was measured to examine whether it may be applied to the bio-medical field. To measure a discharge firing voltage and current and voltage characteristics according to the flow rate of gas, a case where the flow rate of the nitrogen gas was to 4 L/min was experimented. FIG. 5 is a graph illustrating a discharge firing voltage according to a gas flow rate in the plasma jet device of FIG. 1. Referring to FIG. 5, it can be known that the discharge firing voltage of the plasma jet device increases when the gas flow rate increases. The shorter the time the gas stays in an electric field, the higher the voltage required for discharge firing.

### Experimental Example 2

### Discharge Voltage and Current Characteristics

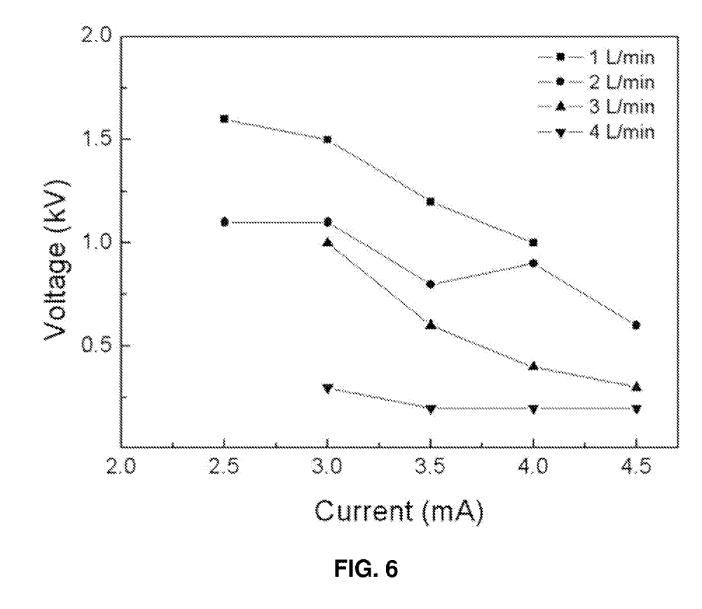

FIG. 6 is a graph illustrating discharge voltage/current characteristics according to a gas flow rate in the plasma jet device of FIG. 1. When a discharge starts, a current rises and the voltage drops. To measure the temperature of jetted plasma, a temperature was measured while jetting plasma to a thin aluminum layer for 10 minutes. The highest temperature of the jetted plasma was 41° C. Since the time of processing plasma, required in bio-medical field, is 10 seconds or so, it is expected that a thermal damage to a cell by the plasma does not occur. In the present embodiment, a device capable of jetting micro plasma under atmospheric pressure was manufactured. The manufactured device successfully jetted plasma under atmospheric pressure. Also, a discharge firing voltage of the device, according to a gas flow rate, was measured and it was not confirmed if the smaller the flow rate, the more discharge firing voltage dropped. Since the highest temperature of the jetted plasma is 41° C., it is regarded as there is no damage on a cell when being applied to the bio-medical field. Hereafter, when optimizing the design of both holes and a thickness of a dielectric layer, it is expected that the atmospheric pressure plasma jet device is capable of being applied to the bio-medical field. As described above, exemplary embodiments have been shown and described. Though specific terms are used herein, they are just used for describing the present invention but do not limit the meanings and the scope of the present invention disclosed in the claims. Therefore, it would be appreciated by those skilled in the art that changes may be made to these embodiments without departing from the principles and spirit of the invention. Accordingly, the technical scope of the present invention is defined by the claims and their equivalents.

### INDUSTRIAL APPLICABILITY

The present invention may be applied to the field of bio-medical.

1. A method of manufacturing an electrode of a plasma jet device, the method comprising:
forming a seed layer on a board;
forming a mold layer on the seed layer;
patterning the mold layer to form a plurality of electrode-forming holes thereon;
forming an electrode layer on the board where the patterned mold layer is formed; and
planarizing the patterned mold layer and the electrode layer; and
removing the board, the seed layer, and the patterned mold layer.

2. The method of claim 1, wherein the seed layer is formed by depositing titanium/gold to a thickness of 500 Å and 2500 Å.

3. The method of claim 2, wherein the titanium/gold is formed by sputtering.

4. The method of claim 1, wherein the mold layer is formed by coating it with a negative sensitizer.

5. The method of claim 4, wherein a thickness of the mold layer is 100 μm or less.

6. The method of claim 1, wherein the mold layer is patterned in such a way that each of the plurality of electrode-forming holes is disposed to be separated from one another at the same interval.

7. The method of claim 6, wherein a width of the electrode-forming holes is 100 μm or less.

8. The method of claim 1, wherein the number of the generated electrode-forming holes is 10×10 or more.

9. The method of claim 1, wherein the electrode layer is formed by plating with a nickel layer.

10. The method of claim 9, wherein a thickness of the nickel layer formed on the electrode-forming holes is 70 μm or less.

11. The method of claim 1, wherein the patterned mold layer and the electrode layer are planarized by chemical mechanical polishing (CMP).

12. The method of claim 11, wherein a thickness of the planarized electrode layer is 60 μm or less.

13. An electrode of the plasma jet device manufactured according to claim 1.

14. A plasma jet device comprising:
the electrode of claim 13, used as an anode, through which plasma is jetted;
a gas injection pipe injecting gas from outside and used as a cathode;
a porous insulating material disposed between the electrode and the gas injection pipe, the porous insulating material insulating the electrode from the gas injection pipe and having a plurality of passing holes to allow the gas injected by the gas injection pipe to be transferred to the electrode;
a protection pipe surrounding the gas injection pipe; and
an insulating case surrounding the porous insulating material to which the electrode and the gas injection pipe are connected and preventing diffusion of a discharge occurring between the electrode and the gas injection pipe to generate the plasma.

15. The plasma jet device of claim 14, wherein the gas injection pipe is formed of stainless steel.

16. The plasma jet device of claim 14, wherein the porous insulating material is formed of ceramic.

17. The plasma jet device of claim 16, wherein the porous insulating material is formed of alumina.

18. The plasma jet device of claim 14, wherein the protection pipe is formed of quartz.

19. The plasma jet device of claim 14, wherein the plasma is jetted to a cell in such a way that the cell kills itself.

20. The plasma jet device of claim 19, wherein the cell killing itself is a cancer cell.
